(19) United States
(12) Patent Application Publication
(10) Pub. No.: US 2023/0363476 A1
(43) Pub. Date: Nov. 16, 2023

(54) FACE MASK

(71) Applicants: THE UNIVERSITY OF BIRMINGHAM, Birmingham (GB); KING'S COLLEGE LONDON, London (GB)

(72) Inventors: Owen Addison, London (GB); Luke Carter, Birmingham (GB); Sophie Cox, Birmingham (GB); Trevor Coward, London (GB); Caroline Reed, London (GB); Liam Grover, Birmingham (GB)

(21) Appl. No.: 18/029,585

(22) PCT Filed: Sep. 22, 2021
(86) PCT No.: PCT/EP2021/076035

§ 371 Date: Mar. 30, 2023

(30) Foreign Application Priority Data
Sep. 30, 2020 (GB) 2015489.4

(51) Int. Cl. A41D 13/11; A61B 5/107

(52) U.S. Cl. CPC A41D 13/1138; A61B 5/1075; A61B 5/1077

(57) ABSTRACT

A method for determining geometry of a face mask comprising a main body and a seal configured to engage a nasal region, cheeks and a chin of a user. The method comprises collecting (402) facial data of the user comprising a point cloud comprising a plurality of points on a skin surface in a nasal region, eye region and chin region of the user; determining (406), based on the facial data, in at least one of the eye region and the chin region of the user, an estimated face size of the user; determining (408), based on the facial data comprising the plurality of points in the nasal region, a best-fit Gaussian curve to a profile of a nose of the user; and using the estimated face size of the user and the determined Gaussian curve to select (410) parameters for a profile geometry of the face mask.

### TECHNICAL FIELD

The invention relates to methods and apparatus for determining geometry of a face mask.

### BACKGROUND

The SARS-CoV-2 virus pandemic has highlighted the need for respiratory personal protective equipment, PPE, that provides effective protection and can be comfortably worn by users over long periods.

Respiratory PPE typically covers at least the nostrils and mouth of a user, and may take the form of a respiratory device or face mask. Respiratory PPE may be required in a number of settings, for example medical settings and construction settings, to protect the wearer from the inhalation of undesired particles. Assuming that an appropriate filtering material is selected, the effectiveness of respiratory PPE may be in large part dictated by the seal that is formed between the respirator device and the face of the user during use. If an effective seal is not formed between the respirator and the face of the user, leaks in the facial seal may occur, exposing the user to the ingress into the face mask of undesired particles, such as airborne pathogens or dust for example. On the other hand, if the seal between the face mask and the face of the user is too tight, the user may experience skin abrasions/irritation, bruising and/or discomfort.

Typically, respiratory devices or face masks may designed as one-size-fits-all devices. The fit of the face mask to the face of the user may be “fit tested” once the user has fitted the mask to their face. Studies have shown that rate of failure of such fit tests is generally high, suggesting that a one-size-fits-all face mask does not provide effective protection for large numbers of the population. The above-mentioned problems associated with leakage and skin abrasions may therefore be commonly experienced by wearers.

As such, there exists a need to provide a face mask of improved fit and comfort to users.

### SUMMARY

According to the invention in a first aspect, there is provided a method for determining a geometry of a face mask comprising a main body and a seal, the seal configured to engage a nasal region, cheeks and a chin of a user, the face mask configured to provide the user with respiratory protection, the method comprising: collecting facial data of the user comprising a point cloud comprising a plurality of points on a skin surface in a nasal region, an eye region and a chin region of the user; determining, based on the facial data comprising the plurality of points in at least one of the eye region and the chin region of the user, an estimated face size of the user; determining, based on the facial data comprising the plurality of points in the nasal region, a best-fit Gaussian curve to a profile of a nose of the user; using the estimated face size of the user and the determined Gaussian curve to select parameters for a profile geometry of the face mask.

Optionally, the estimated face size is used to select parameters for a size of the seal and/or the main body.

Optionally, the Gaussian curve is used to select parameters for a nasal profile geometry of the seal and/or the main body.

Optionally, determining the estimated face size comprises determining a scaling factor indicative of a size of a face of the user, based on the facial data.

Optionally, determining the scaling factor comprises determining a distance between facial features of the user, based on the facial data.

Optionally, the facial features comprise an eye level of the user and the chin of the user.

Optionally, the method further comprises determining, from the facial data, a nasal ridge plane defining a gradient of a nasal ridge of the user.

Optionally, the best-fit Gaussian curve is determined to the profile of the nose of the user in a plane transverse to the nasal ridge plane.

Optionally, selecting the parameters for the profile geometry of the face mask comprises selecting one of a plurality of predetermined face masks.

Optionally, the one of the plurality of predetermined face masks that comprises geometry that most closely corresponds to the estimated face size and/or the determined Gaussian curve is selected from the plurality of face masks.

Optionally, the method further comprises performing regression analysis to determine the one of the plurality of predetermined face masks.

Optionally, the method further comprises determining whether the estimated face size of the user and the determined Gaussian curve are within a threshold range.

Optionally, the one of the plurality of predetermined face masks is selected if the estimated face size of the user and the determined Gaussian curve fall within the threshold range.

Optionally, if the estimated face size of the user and the determined Gaussian curve fall outside of the threshold range, one of the plurality of predetermined face masks is not selected, and the method further comprises selecting the parameters for the profile geometry of the face mask using geometry defined by the point cloud.

Optionally, selecting the parameters for the profile geometry comprises modifying parameters of one of the plurality of predetermined face masks based on the geometry defined by the point cloud.

Optionally, selecting the one of the plurality of predetermined face masks comprises selecting one of a plurality of predetermined main bodies and/or selecting one of a plurality of predetermined seals.

Optionally, determining the best-fit Gaussian curve comprises determining a width and height of the profile of the nose, based on the facial data, and selecting the parameters of the Gaussian curve based on the determined width and height.

Optionally, the method further comprises orientating the point cloud based on one or more facial features in the point cloud.

Optionally, the facial features comprise an eye level and a tip of a chin of the user. Optionally, orientating the point cloud comprises determining an eye level and a tip of a chin of the user based on the facial data, and orientating the point cloud based on an eye level plane defined by the eye level and an eye-to-chin plane defined by the eye level and the tip of the chin.

Optionally, the point cloud is orientated such that the eye level plane is transverse to the eye-to-chin plane.

Optionally, orientating the point cloud further comprises extracting a side profile of a face of the user, using the facial data.

Optionally, the nasal ridge plane is determined by selecting a subset of the plurality of points of the point cloud of the nasal ridge, based on the side profile, and defining the nasal ridge plane based on a line of best fit determined using the subset of the plurality of points.

Optionally, the subset of the plurality of points are separated by a predetermined distance along an axis defined between the eye level and the tip of the chin.

Optionally, collecting the facial data of the user comprises performing a 3-dimensional, 3D, scan.

Optionally, the face mask comprises a particle filtering half mask.

Optionally, the particle filtering half mask comprises a filtering facepiece 3, FFP3, respirator.

Optionally, the particle filtering half mask is configured to provide protection against respiratory borne pathogens.

According to the invention in a further aspect, there is provided a system for determining a geometry of a face mask comprising a main body and a seal, the seal configured to engage a nasal region, cheeks and a chin of a user, the face mask configured to provide the user with respiratory protection, the system comprising: a facial data collector configured to collect facial data of the user comprising a point cloud comprising a plurality of points on a skin surface in a nasal region, an eye region and a chin region of the user; and an apparatus comprising: a receiver configured to receive the facial data from the facial data collector; a face size estimator configured to determine, based on the facial data comprising the plurality of points in at least one of the eye region and the chin region of the user, an estimated face size of the user; a nasal profile determiner configured to determine, based on the facial data comprising the plurality of points of the nasal region, a best-fit Gaussian curve to a profile of a nose of the user; and a mask geometry determination unit configured to select parameters for a profile geometry of the face mask using the estimated face size of the user and the determined Gaussian curve.

Optionally, the system further comprises a plurality of predetermined face masks.

Optionally, the system further comprises at least one of a plurality of predetermined main bodies and a plurality of predetermined seals.

According to the invention in a further aspect, there is provided a computer program comprising instructions which, when executed on at least one processor, cause the at least one processor to carry out the method according to any one of claims 1 to 22.

According to the invention in a further aspect, there is provided a face mask comprising a main body and a seal, the seal configured to engage a nasal region, cheeks and a chin of a user, the face mask configured to provide the user with respiratory protection, the face mask obtainable by the method according to any one of claims 1 to 22.

### BRIEF DESCRIPTION OF THE DRAWINGS

FIG. 1 is a schematic view of an exemplary system for determining geometry of a face mask;

FIG. 2 is a schematic representation of an exemplary apparatus for determining geometry of a face mask;

FIG. 3 is a side view of an exemplary face mask;

FIG. 4 is a flow diagram of an exemplary method for determining geometry of a face mask;

FIG. 5 shows an exemplary 2D profile extracted from a 3D model of a face of a user;

FIG. 6 shows an exemplary 2D profile extracted from a 3D model of a face of a user;

FIG. 7 is a flow diagram of an exemplary method for determining profile geometry of a face mask;

FIG. 8(a) shows an exemplary 3D model of a face of a user; and

FIG. 8(b) shows an exemplary 2D profile extracted from a 3D model of a face of a user.

### DETAILED DESCRIPTION

Generally disclosed herein are methods and apparatus for determining a geometry of a face mask. The face mask may be a half mask. That is, a mask which covers only part of the face. Specifically, the face mask may be a mask that covers the nostrils and mouth of a user or wearer, but not other orifices, such as the eyes.

Exemplary methods may comprise collecting facial data, extracting parameters from the facial data, and selecting parameters for a profile geometry of the face mask based on the extracted parameters. Exemplary methods comprise estimating a face size of the user and a profile of the nose of the user based on the extracted parameters. The estimated face size may be used to select parameters for a size of the face mask, and the determined profile of the nose of the user may be used to select parameters for a nasal profile geometry of the face mask.

In exemplary methods, the facial data may comprise a point cloud comprising a plurality of points on a skin surface of a user in a nasal region, eye region and chin region of the user. The face size of the user may be estimated using the plurality of points of the point cloud in at least one of the eye region and the chin region. The profile of the nose of the user may be estimated using the plurality of points of the point cloud in the nasal region. In exemplary methods, selecting the parameters for the nasal profile of the face mask comprises determining a best-fit Gaussian curve to a profile of a nose of the user.

The inventors have realised that while not all of the population have nasal profiles that correspond to a Gaussian curve, most nasal profiles may be approximated to a Gaussian curve within +/−1 mm. This discrepancy may be accounted for by appropriate material selection for the face mask (for example, material with sufficient elasticity within the portion of the face mask configured to contact the skin surface of the user) and the elasticity of the tissue of the nose. As such, by determining a nasal profile geometry of the face mask based on a best-fit Gaussian curve, an improved seal in the nasal region may be provided. This reduces leakage around the nasal region.

Further, the force required to be applied to the face mask to hold it flush against the face of the user is reduced. Known face masks may comprise gasket-style seals, which rely on sufficient force being applied to the face mask to deform the gasket-style seal to the wearer's face. The increased force required to deform the gasket-style seal may cause abrasions and/or bruising, particularly in areas of the face with reduced soft tissue, such as the nose and chin. Using the exemplary methods described within this application, an improved fit face mask may be produced. In exemplary methods the parameters selected for the profile geometry of the face mask are based on the user's facial data, and as such, the portion of the face mask configured to contact the user's skin more accurately corresponds to the profile of the user's face. As such, a more effective seal is created between the face mask and the user's face, therefore reducing leakage. Furthermore, a more comfortable fit is provided. This is because the profile of the seal largely corresponds to the user's face without large amounts of force having to be applied to the face mask, for example by the head straps. As such, abrasions and bruising may be reduced particularly in those areas of the face where there is a lack of soft tissue, for example in the nasal region.

In particular, the methods described below may provide a face mask with improved fit in the nasal region of a user, which the inventors have identified as being a problematic area for leakage.

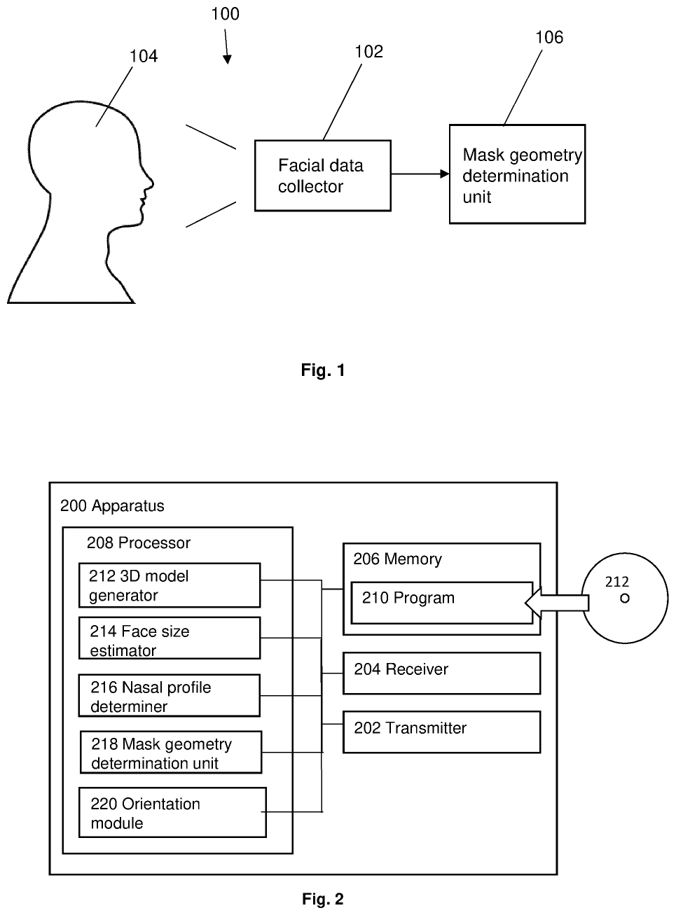

FIG. 1 shows an exemplary system 100 for determining a geometry of a face mask. The system 100 comprises a facial data collector 102 configured to collect facial data from a user 104. The user 104 may be the eventual wearer of the face mask. The facial data collector 102 may be configured to collect facial data comprising a point cloud comprising a plurality of points on a skin surface of the face of the user 104.

The facial data may therefore be indicative of facial features of the user. For example, the facial data may comprise data indicative of one or more of a shape, size, contour or profile of the face of the user and/or facial features of the user.

In exemplary methods, each of the plurality of points of the point cloud may comprise co-ordinates along an x, y and z axis. Each of the plurality of points may optionally further comprise one or more of a vector, colour value (such as an RBG value) and an intensity value indicative of how bright the point is.

The skilled person will appreciate that there are many ways to collect the facial data of the user 104 comprising the point cloud. For example, in exemplary arrangements, the facial data collector 102 may comprise a scanner, such as a 3D scanner. The 3D scanner may be configured to capture the facial data using non-contact methods such as lasers, or contact methods such as contact probes to create the point cloud of data from the skin surface of the user. Alternatively or additionally, the facial data collector 102 may comprise a camera configured to collect images of the face of the user, and photogrammetry may be used to create the point cloud. The skilled person will be able to envisage further arrangements.

Exemplary systems may further comprise a plurality of predetermined face masks. Exemplary systems may further comprise a plurality of predetermined seals and/or a plurality of predetermined main bodies of a face mask, as will be described in more detail below.

The system 100 may further comprise an apparatus 106 configured to determine the geometry of the face mask based on the facial data collected by the facial data collector 102. The apparatus may comprise a mask geometry determination unit. The apparatus 106 may comprise a user device, such as a computing device, a laptop, a mobile device such as a mobile phone or a tablet. The skilled person will be able to envisage further user devices.

FIG. 2 shows a schematic representation of an exemplary apparatus 200 for determining the geometry of a face mask, which may be the apparatus 106. The apparatus 200 may comprise a transmitter 202 and/or a receiver 204. The transmitter 202 and/or the receiver 204 may be in data communication with other entities such as user devices, servers and/or other devices, apparatus or systems. In exemplary arrangements the receiver 204 may receive facial data from the facial data collector 102. The transmitter 202 and/or the receiver 204 may be configured to transmit and receive data accordingly.

The apparatus 200 may further comprise a memory 206 and a processor 208. The memory 206 may comprise a non-volatile memory and/or a volatile memory. The memory 206 may have a computer program 210 stored therein. The computer program 210 may be configured to undertake the methods disclosed herein. The computer program 210 may be loaded in the memory 206 from a non-transitory computer readable medium 212, on which the computer program is stored. The processor 208 may be configured to undertake one or more of the functions of a 3D model generator 212, a face size estimator 214, a nasal profile determiner 216, a mask geometry determination unit 218, and an orientation module 220, as set out below.

Each of the transmitter 202 and receiver 204, memory 206, processor 208, 3D model generator 212, a face size estimator 214, a nasal profile determiner 216, a mask geometry determination unit 218, and an orientation module 220 may be in data communication with the other features 202, 204, 206, 208, 210, 212, 214, 216, 218, 220 of the apparatus 200. The apparatus 200 may be implemented as a combination of computer hardware and software. In particular, the 3D model generator 212, face size estimator 214, nasal profile determiner 216, mask geometry determination unit 218, and orientation module 220 may be implemented as software configured to run on the processor 508. The memory 206 may store the various programs/executable files that are implemented by the processor 208, and may also provide a storage unit for any required data. The programs/executable files stored in the memory 206, and implemented by the processor 208, can include the 3D model generator 212, face size estimator 214, nasal profile determiner 216, mask geometry determination unit 218, and orientation module 220, but are not limited to such.

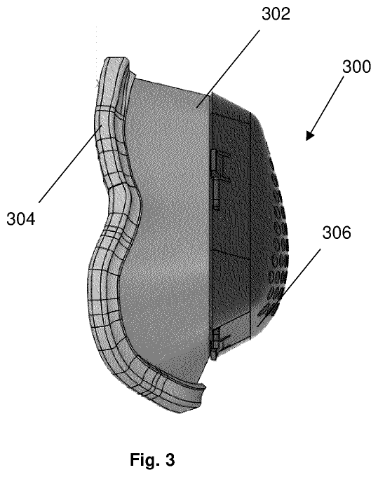

To aid understanding, FIG. 3 shows a side view of an exemplary face mask 300 and features thereof. Exemplary methods described herein may be used to determine a geometry of a face mask, such as the face mask 300. However, the skilled person will appreciate that the face mask 300 is exemplary only, and that the methods disclosed herein could also be used to determine geometries of alternative face masks, with alternative features.

The exemplary face mask 300 may be a half mask. That is, the exemplary face mask 300 may be configured to extend over a portion of a face of the wearer. The exemplary face mask 300 may be configured to extend over a lower portion of the face of the wearer. In exemplary arrangements, the face mask 300 may an oro-nasal half mask. That is, the face mask may be configured to cover nostrils and a mouth of the user but not extend to cover other orifices such as the eyes. The face mask 300 may be a reusable face mask. That is, a face mask configured to be used for more than a single shift.

The face mask 300 may comprise a particle filtering face mask. The face mask 300 may be configured to provide protection against undesired particles such as respiratory borne pathogens, solid particles, for example dust, and particles of/carried in liquids and/or chemicals. The face mask 300 may be comprise a filtering facepiece, FFP, mask configured to meet the requirements of an FFP1, FFP2 or FFP3 face mask, as defined in the European Standard EN-149.

The face mask 300 shown in FIG. 3 comprises a main body 302 and a seal 304. The main body 302 may be configured to enclose the lower portion of the face of the wearer. In the exemplary arrangement, the seal 304 extends around a perimeter of the main body 302. The seal 304 may be configured to contact a skin surface of the wearer's face to prevent ingress of airborne particles into the main body 302.

The face mask 300 may further comprise a filter 306. The filter 306 may comprise filter material and a filter cap configured to retain the filter material. The filter 306 may be configured to prevent the ingress of particles into the main body 302 on an inhale of a user. In exemplary arrangements, the filter material may be configured to prevent the ingress of undesired particles. The filter material may be configured to trap and/or retain the undesired particles.

As outlined above, the effectiveness of a face mask, such as the face mask 300, may be in large part dictated by the seal that is formed between the face mask 300 and the user's face during use. An improved seal may be formed by determining geometry of the face mask, such as geometry of the main body 302 and/or the seal 304, based on facial characteristics of the user.

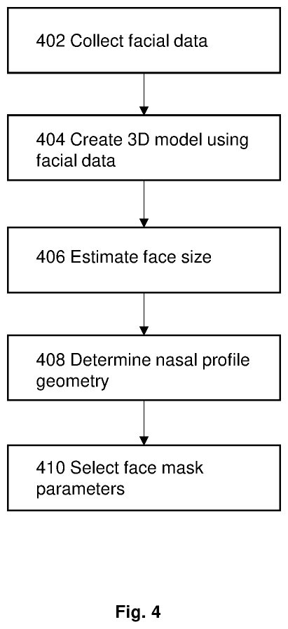

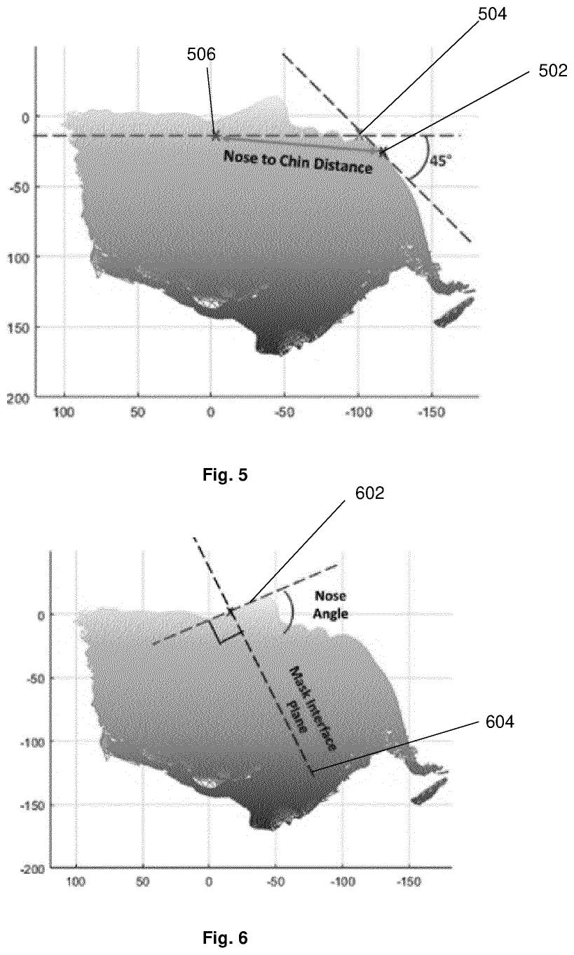

FIG. 4 shows a flow diagram of an exemplary method for determining a geometry of a face mask. The face mask may comprise the face mask 300 described above. The steps of the method are described below with reference to FIGS. 1-6.
402: Facial data is collected from the user 104, by the facial data collector 102. In exemplary arrangements, the facial data may comprise a point cloud comprising a plurality of points on a skin surface of the face of the user.
In exemplary arrangements, the point cloud may comprise a plurality of points on the skin surface of the user over substantially the whole of the user's face. In exemplary methods, the point cloud may comprise a plurality of points on a skin surface of the user in at least one of a nasal region, an eye region and a chin region of the user.
The nasal region may comprise at least the nose of the user. The eye region may comprise at least one eye of the user and features thereof. In exemplary methods, the eye region may comprise the periorbital region. For example, the eye region may comprise one or more of: the eye of the user; the eye lid(s) of the user; and the eyebrow of the user. The eye region my comprise both eyes of the user. The chin region may comprise the chin of the user. The chin region may further comprise one or more of: a mouth of the user; a maxilla of the user; and a mentolabial sulcus (the indentation that separates the lower lip from the tip of the chin).
404: A 3D model of the face of the user 104 may be created based on the facial data, by a 3D model generator 212. The 3D model may comprise the plurality of points of the point cloud. In alternative methods, a 3D model may not be created, and the further method steps outlined below may be completed based on the point cloud without conversion to a 3D model.
The skilled person will appreciate that there are many ways to create a 3D model based on the point cloud. For example, a 3D model may be directly rendered based on the point cloud. Alternatively, a mesh model may be generated based on the point cloud. The mesh may then be used to generate the 3D model. The skilled person will be familiar with the methods and software that may be used to generate a 3D model based on the point cloud.
Since the generated 3D model is a model of the face of the user, facial features of the user may be identified within the 3D model. For example, nasal characteristics, such as shape, size and/or profile of the nose, may be identifiable from the 3D model. As such, for the purposes of the description below, facial features “of the 3D model” or “of/within the point cloud” are discussed. The skilled person will appreciate that facial features of/within the 3D model/point cloud encompass the 3D model representation/point cloud representation of the corresponding facial feature of the user. For example, the nose of the 3D model means the portion of the 3D model corresponding to the nose of the user.
406: A size of the face of the user 104 is estimated by the face size estimator 214, based on the facial data. The size of the face of the user may encompass one or more of a height, a width, an aspect ratio, and a perimeter of the face of the user.
A size of the face mask may be selected based on the estimated face size of the user. The size of the face mask may encompass one or more of a height, a width, an aspect ratio, and a perimeter of the face mask.
In exemplary methods, estimating the face size of the user 104 may comprise determining a scaling factor. The scaling factor may comprise a distance between facial features of the user, determined based on the facial data. The scaling factor may be indicative of facial height of the user. In alternative arrangements, the scaling factor may be indicative of facial width of the user.
In exemplary methods, determining the scaling factor may comprise identifying facial features, based on the facial data. In exemplary methods, facial features may be identified using facial recognition methods. In alternative methods, facial features may be identified manually by a user selecting points within the 3D model. The facial features may be representative of the points and/or regions between which the face mask is to extend or which the face mask is configured to contact. Identifying the facial features may comprise identifying one or more of: an eye level; a chin region; a tip of a chin; a mouth; a nasal region; a tip of a nose; a cheek of a user.
In exemplary methods, in which the face mask is configured to extend between the bridge of the nose and the chin of the user, the scaling factor may comprise a determined distance between the bridge of the nose and a chin of the user. The bridge of the nose may be determined based on an eye level of the user. The method may comprise identifying one or more of: an eye level, a bridge of the nose of the user and a top of the chin of the user, based on the facial data.
Identifying the eye level of the user may comprise identifying one or more features of an eye of the user from the facial data. For example, in exemplary methods, the eye level of the user may be defined by the inner corners of the eyes (the inner canthus of the eyes). As such, identifying the eye level of the user may comprise identifying the inner corners of the eyes, based on the facial data. In alternative methods, the eye level may be estimated by identifying alternative features of at least one eye of a user, based on the facial data. For example, an outer corner of the eye(s).
Identifying the tip of the chin of the user may comprise using facial recognition, or alternatively manual selection, as discussed above.
A further exemplary method of identifying the tip of the chin is described below with reference to FIG. 5.
The tip of the chin 502 may be identified based on a reference point 504. The reference point 504 may be located in the chin region of the point cloud. In exemplary methods, the reference point 504 may be selected by a user. The reference point 504 may be located on a flat of the chin. The flat of the chin may encompass a region of the chin at substantially a maxima of a curve of the chin. That is, the maximum of the curve of the chin when a face is viewed in side profile.
In exemplary methods, a 2D side profile, as shown in FIG. 5, may be extracted from the 3D model to aid identification of the flat of the chin and/or identification of further facial features.
In exemplary methods, the tip of the chin 502 may be determined, based on the reference point 504. The tip of the chin 502 may be determined based on a predefined relationship with respect to the reference point 504.
The tip of the chin may be defined as the point within the point cloud located at a predetermined angle from the reference point 504. FIG. 5 shows the tip of the chin 502 as the point in the point cloud located at 45 degrees from the reference point 512, however the skilled person will appreciated that alternative angles may be selected. Specifically, the tip of the chin may be determined as the point in the point cloud located at 45 degrees from an axis defined by the flat of the chin and the inner corner of an eye of the user 506. Again, however, the skilled person will appreciate that alternative axes may be defined.
Determining the scaling factor may comprise determining a distance between the identified facial features. In exemplary methods, determining the scaling factor may comprise determining the distance between the identified eye level and the identified tip of the chin. In alternative methods, determining the scaling factor may comprise determining the distance between the eye level and the mentolabial sulcus (the indentation that separates the lower lip from the chin).
The scaling factor may be used to select parameters for a size of the face mask, as will be described in more detail below.
408: A nasal profile geometry of the face mask is determined by a nasal profile determiner 216, based on the facial data.
Determining the nasal profile geometry of the face mask may comprise determining a best-fit Gaussian curve to a profile of a nose of the user, based on the facial data. In exemplary arrangements, the best-fit Gaussian curve may be determined based on the plurality of points of the point cloud in the nasal region.
A Gaussian curve may be defined by the relationship:

$$f ( x ) = {Ae}^{- \frac{{x}^{2}}{2 {σ}^{2}}} ,$$

where A is the height of the peak of the curve, and a is the width of the curve.
The skilled person will be familiar with methods used to determine a best-fit Gaussian curve to a set of data points. Exemplary methods may comprise using regression methods to determine the best-fit Gaussian curve. In exemplary methods, the regression method may comprise least squares methods. Least squares fitting may comprise fitting a polynomial to the set of data points. Parameters of the Gaussian curve may be determined based on extracted coefficients of the fitted polynomial. For example, a height and width of the Gaussian curve may be determined based on the extracted coefficients of the fitted polynomial indicating a peak of the polynomial curve and a maximum width. In alternative exemplary methods, alternative fitting methods may be used to determine the best-fit Gaussian curve. For example, trial and error methods may be used. Alternatively, a width and height of a nose profile at a given position may be directly extracted from the facial data and used to select a width and height of the Gaussian curve.
The inventors have realised that the fit of the nasal profile of the face mask to a user may be improved by fitting the Gaussian curve to the profile of the nose of the user in a plane representative of the angle at which the face mask contacts the nose of the wearer. As such, exemplary methods may comprise determining the best-fit Gaussian curve in a mask interface plane disposed at an angle to a nasal ridge plane. For example, exemplary face masks may contact the nose of a wearer in a plane substantially transverse to the nasal ridge plane. As such, exemplary methods may comprise determining the best-fit Gaussian curve in a plane substantially transverse to the nasal ridge plane. FIG. 6 shows an exemplary nasal ridge plane 602 and a mask interface plane 604 substantially transverse to the nasal ridge plane 602. The best-fit Gaussian curve may determined in the mask interface plane 604.
Within this application, the skilled person will understand that the nasal ridge plane encompasses a plane defined by a gradient of a nasal ridge of the user. The nasal ridge may encompass a midline of the nose of the user extending between the bridge of the nose and the tip of the nose. The bridge of the nose may comprise the saddle-shaped area of the nose, located between the eyes. As such, thebridge of the nose may be located at substantially eye level.
In exemplary methods, the nasal ridge plane may be defined by a gradient determined based on substantially the whole of the nasal ridge. In alternative methods, the nasal ridge plane may be defined by a gradient determined based on at least of portion of the nasal ridge of the user.
In exemplary methods, a plurality of points along the nasal ridge may be determined, and the nasal ridge plane may be defined by a line of best fit determined based on the plurality of points. The plurality of points may comprise two points separated by a predetermined distance along the nasal ridge. For example, in exemplary methods, the nasal ridge plane may be defined by a pair of points on the nasal ridge separated by substantially 5 mm, substantially 8 mm, substantially 10 mm, substantially 12 mm, and substantially 15 mm. The skilled person will appreciate however that substantially any number of points may be used to determine the nasal ridge plane. In exemplary methods, the plurality of points may be selected along a predetermined distance of the nasal ridge. For example, the plurality of points may be selected from within a substantially 5 mm distance along the nasal ridge; within a substantially 8 mm distance along the nasal ridge; within a substantially 10 mm distance along the nasal ridge; within a substantially 12 mm distance along a nasal ridge; and within a substantially 15 mm distance along the nasal ridge.
In exemplary methods, the nasal ridge plane may be determined based on a plurality of points located in an upper region of the nasal ridge. The upper region of the nasal ridge may extend from the bridge of the nose. The upper region may encompass an upper half of the nasal ridge; an upper third of the nasal ridge and an upper quarter of the nasal ridge.
Identification of the nasal ridge within the model may encompass any of the methods outlined above for determining facial features based on the facial data. For example, facial recognition methods or manual selection by the user. In alternative methods, a 2D profile may be extracted from the 3D model. The 2D profile, such as the profile shown in FIGS. 5 and 6, may be extracted along a midline of the face of the user. The nasal ridge may be determined based on the side profile of the nose defined by the 2D profile, see for example FIG. 6.
410: Profile geometry of the face mask is determined, by the mask geometry determination unit 218, based on the estimated face size and the determined
Gaussian curve.
In exemplary methods, parameters for the size of the face mask may be selected based on the estimated face size. For example, one or more of a height, width, aspect ratio or perimeter of the face mask may be selected based on the scaling factor.
Parameters for the nasal profile of the face mask may be selected based on the determined best-fit Gaussian curve.

The above-mentioned method provides an improved fit face mask.

The inventors have realised that providing a fully customised face mask for each user, based on the facial data, may require large amounts of computing resources, and have associated time overheads that do not allow face masks to be produced in numbers great enough, or quickly enough, to meet demand.

The inventors have further realised that a predetermined number of face masks comprising predetermined sizes and nasal profiles may provide a sufficient fit for large numbers of the population, eliminating the need for time consuming and resource intensive customisation processes and manufacture. In exemplary methods, one of a plurality of predetermined face masks may be selected based on the estimated face size and determined Gaussian curve. In exemplary methods, the one of the plurality of predetermined face masks comprising a size and nasal profile geometry that is closest to the estimated face size of the user and the determined best-fit Gaussian curve may be selected.

By selecting face mask profile geometry based on limited features determined from the captured facial data (such as the scaling factor and the best-fit Gaussian curve), a much more rapid evaluation of the facial data may be performed, which reduces computational burden. This is particularly advantageous when compared to fully customised methods that match the contour of the face mask to the contours, shapes and sizes of the facial features of the user. As such, in exemplary methods, the profile geometry of the face mask may be selected based only on the estimated face size and the determined Gaussian curve.

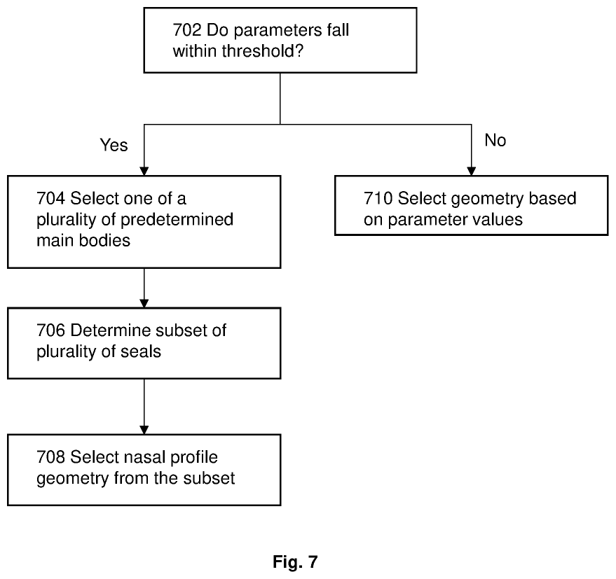

FIG. 7 is a flow diagram showing an exemplary method for selecting face mask geometry by selecting one of a plurality of predetermined face masks. Selecting one of a plurality of predetermined face masks may comprise selecting one of a plurality of predetermined main bodies 302 and one of a plurality of predetermined seals 304.
702: The mask geometry determination unit may determine whether the at least one of the estimated face size and the best-fit Gaussian curve fall within a threshold range. If the estimated face size of the user and/or the determined Gaussian curve fall within the threshold range, one of the plurality of predetermined face masks may be selected. In exemplary methods, one of the plurality of predetermined face masks may be selected if both the estimated face size and the determined Gaussian curve fall within the threshold range. In alternative methods, one of the plurality of predetermined face masks may be selected if at least one of the estimated face size and the determined Gaussian curve fall within the threshold range
If the estimated face size of the user and/or the determined Gaussian curve fall outside of the threshold range, one of the plurality of predetermined face masks may not be selected, and the geometry of the face mask may be selected using the estimated face size and determined Gaussian curve values. In exemplary methods, one of the plurality of predetermined face masks may not be selected if both the estimated face size and the determined Gaussian curve fall outside the threshold range. In alternative methods, one of the plurality of predetermined face masks may not be selected if at least one of the estimated face size and the determined Gaussian curve fall outside the threshold range
704: If at least one of the estimated face size of the user and the determined Gaussian curve fall within the threshold range, one of a plurality of predetermined main bodies is selected based on the estimated face size. One of the plurality of predetermined main bodies may be selected based on the one of the plurality of predetermined main bodies comprising geometry most closely corresponding to the estimated face size.
The skilled person will understand that there are many methods that may be used to determine which of the plurality of predetermined main bodies comprises a size that most closely corresponds to the estimated face size of the user. For example, exemplary methods may comprise performing regression analysis.
In exemplary methods, selecting the one of the plurality of main bodies may comprise selecting one of the plurality of predetermined main bodies with a parameter that most closely matches the determined scaling factor. For example, if the scaling factor comprises the distance between the eye level and the tip of the chin of the user, the predetermined main body with a height that most closely matches the scaling factor may be selected. If the scaling factor comprises the distance between the cheeks of the user, the predetermined main body with a width that most closely matches the scaling factor may be selected. The skilled person will be able to envisage further scaling factors and corresponding main body parameters.
706: In exemplary methods, a subset of a plurality of predetermined seals may be determined by the mask geometry determination unit 218.
The subset of the plurality of seals may be determined based on the size of the selected main body. In alternative methods, the subset of the plurality of seals may be determined based on the estimated face size of the user.
Each of the plurality of seals within the subset may comprise a size corresponding to the size of the selected main body, or the estimated face size of the user, but comprise different nasal profiles.
708: One of the subset of the plurality of predetermined seals is selected based on the determined best-fit Gaussian curve. The one of the subset of the plurality of seals may be determined based on the one of the subset of the plurality of seals comprising nasal profile geometry that most closely matches the best-fit Gaussian curve.
As discussed above, the skilled person will understand that there are many methods that may be used to determine which one of the subset of the plurality of predetermined seals comprises a nasal profile geometry that most closely corresponds to the determined best-fit Gaussian curve. For example, exemplary methods may comprise performing regression analysis.
A face mask may be formed based on the selected one of the plurality of predetermined main bodies and the one of the plurality of predetermined seals.
710: Although a large proportion of the population may achieve a satisfactory face mask fit based on one of the plurality of predetermined main bodies and seals, there may be some for who the predetermined face masks are not appropriate.
If the estimated face size of the user and the determined Gaussian curve do not fall within the threshold range, one of the predetermined face masks may not be selected. Profile geometry of the face mask may instead be determined using the values for the estimated face size and the best-fit Gaussian curve.
For example, a predetermined face mask may be modified based on the scaling factor determined by the face size estimator. For example, where the scaling factor comprises the distance between the eye level and chin tip of the user, and so is indicative of a desired height of the face mask, the predetermined face mask may be scaled such that the height corresponds to the scaling factor. Other parameters of the predetermined face mask, such as width, may be scaled accordingly.
The nasal profile of the predetermined face mask may be modified to correspond to the dimensions defined by the best-fit Gaussian curve.

In exemplary methods selecting the profile geometry of the face mask may further comprise defining a minimum thickness for a seal of the face mask. In exemplary methods, selecting a minimum thickness for the seal may comprise defining a minimum thickness of a portion of the seal configured to extend between the main body and the face of the user, when the mask is in use.

The inventors have realised that the portions of the seal configured to contact areas of the face with reduced soft tissue, such as the nose and chin, may benefit from increased seal thickness. This is because the seal is typically formed of from a material, such as silicone, which has greater elasticity than the main body of the face mask. This further reduces abrasions and bruising caused by the face mask.

Exemplary methods may therefore comprise defining a minimum thickness for the seal. The minimum thickness of the portion of the seal configured to extend between the main body and the face of the wearer may be one of substantially 3 mm, 4 mm, 5 mm, 6 mm, and 7 mm.

The portion of the seal configured to extend between the main body and the face of the wearer in the nasal region may comprise a thickness greater than the minimum thickness. In exemplary face masks, the portion of the seal configured to extend between the main body and the face of the wearer in the chin region may comprise a thickness greater than the minimum thickness. In exemplary face masks, the portion of the seal configured to extend between the main body and the face of the wearer in a maxilla region may comprise a thickness greater than the minimum thickness.

For example, an exemplary face mask may comprise a minimum thickness of substantially 5 mm. That is, the minimum thickness of the portion of the seal configured to extend between the main body and the skin surface of the wearer may be substantially 5 mm. The thickness of the portion of the seal configured to extend between the main body and the face of the wearer in the nasal region may be greater than 5 mm, for example, substantially 6 mm. The thickness of the portion of the seal configured to extend between the main body and the face of the wearer in the chin region may be greater than 5 mm, for example, substantially 9 mm. The thickness of the portion of the seal configured to extend between the main body and the face of the wearer in the chin region may be greater than 5 mm, for example, substantially 7 mm.

The above example is exemplary only and the skilled person will be able to envisage further arrangements.

In exemplary methods, extraction of the above-mentioned parameters (for example, the scaling factor and the Gaussian curve) may be facilitated by orientating, by an orientation module 220, the 3D model. The skilled person will appreciate however that this is an optional process and will be able to envisage methods of extracting the parameters without orientation of the 3D model, as described above.

Orientating the 3D model may comprise aligning one or more facial features of the 3D model with one of an x, y and z axis. For example, the eye level and the midline of the face may be orientated with perpendicular axes.

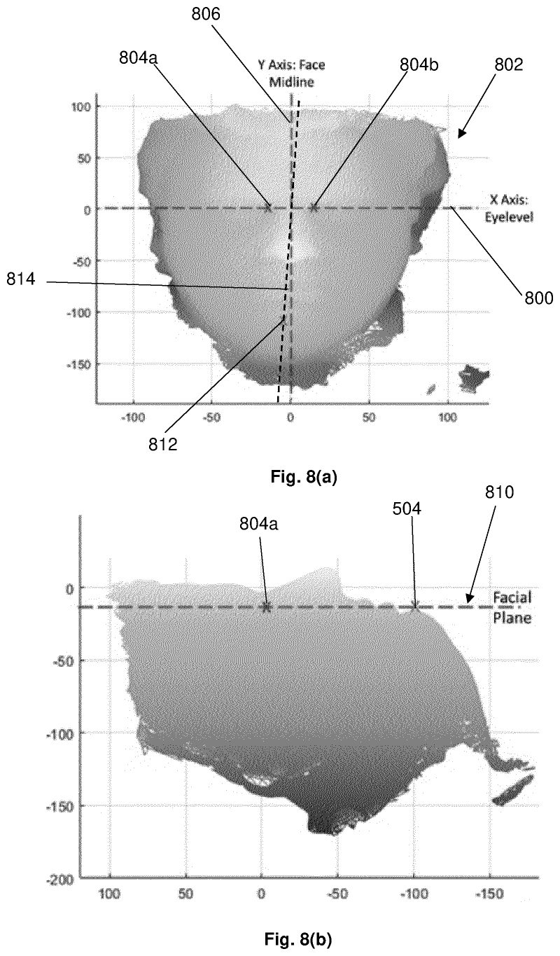

In exemplary methods, orientating the 3D model may comprise defining the eye level of the 3D model as a first axis 800, as shown in FIG. 8(a). The first axis 800 may be the x-axis. As described above, the eye level of the 3D model 802 may be defined by the inner corners 804a, 804b of the eyes (or inner canthi). The 3D model may be orientated such that the first axis 800 intersects the points defined by the inner corners 804a, 804b. In exemplary methods, the inner corners of the eyes 804a, 804b may be manually identified by a user.

Orientating the 3D model may further comprise defining a midline of the face represented by the 3D model as a second axis 806, perpendicular to the first axis 800. The second axis 806 may be the y-axis.

Orientating the 3D model may further comprise aligning a facial plane 810 with a plane defined by the first and second axes, as shown in FIG. 8(b). The facial plane may be defined by the inner corners of the eyes 804a, 804b and a flat of the chin 812. In exemplary methods, the flat of the chin 812 may be manually identified by a user.

The skilled person will appreciate that orientating the 3D model may comprise defining the axes based on alternative facial features to the ones mentioned above.

A computer program may be configured to provide any of the above described methods. The computer program may be provided on a computer readable medium. The computer program may be a computer program product. The product may comprise a non-transitory computer usable storage medium. The computer program product may have computer-readable program code embodied in the medium configured to perform the method. The computer program product may be configured to cause at least one processor to perform some or all of the method.

Various methods and apparatus are described herein with reference to block diagrams or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

Computer program instructions may also be stored in a computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks.

A tangible, non-transitory computer-readable medium may include an electronic, magnetic, optical, electromagnetic, or semiconductor data storage system, apparatus, or device. More specific examples of the computer-readable medium would include the following: a portable computer diskette, a random access memory (RAM) circuit, a read-only memory (ROM) circuit, an erasable programmable read-only memory (EPROM or Flash memory) circuit, a portable compact disc read-only memory (CD-ROM), and a portable digital video disc read-only memory (DVD/Blu-ray).

The computer program instructions may also be loaded onto a computer and/or other programmable data processing apparatus to cause a series of operational steps to be performed on the computer and/or other programmable apparatus to produce a computer-implemented process such that the instructions which execute on the computer or other programmable apparatus provide steps for implementing the functions/acts specified in the block diagrams and/or flowchart block or blocks.

Accordingly, the invention may be embodied in hardware and/or in software (including firmware, resident software, micro-code, etc.) that runs on a processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated.

The skilled person will be able to envisage other embodiments without departing from the scope of the appended claims.

1. A method for determining a geometry of a face mask comprising a main body and a seal, the seal configured to engage a nasal region, cheeks and a chin of a user, the face mask configured to provide the user with respiratory protection, the method comprising:
collecting facial data of the user comprising a point cloud comprising a plurality of points on a skin surface in a nasal region, an eye region and a chin region of the user;
determining, based on the facial data comprising the plurality of points in at least one of the eye region and the chin region of the user, an estimated face size of the user;
determining, based on the facial data comprising the plurality of points in the nasal region, a best-fit Gaussian curve to a profile of a nose of the user;
using the estimated face size of the user and the determined Gaussian curve to select parameters for a profile geometry of the face mask.

2. A method according to claim 1, wherein the estimated face size is used to select parameters for a size of the seal and/or the main body.

3. A method according to claim 1, wherein the Gaussian curve is used to select parameters for a nasal profile geometry of the seal and/or the main body.

4. A method according claim 1, wherein determining the estimated face size comprises determining a scaling factor indicative of a size of a face of the user, based on the facial data.

5. A method according to claim 4, wherein determining the scaling factor comprises determining a distance between facial features of the user, based on the facial data.

6. A method according to claim 5, wherein the facial features comprise an eye level of the user and the chin of the user.

7. A method according to claim 1, further comprising determining, from the facial data, a nasal ridge plane defining a gradient of a nasal ridge of the user.

8. A method according to claim 7, wherein the best-fit Gaussian curve is determined to the profile of the nose of the user in a plane transverse to the nasal ridge plane.

9. A method according to claim 1, wherein selecting the parameters for the profile geometry of the face mask comprises selecting one of a plurality of predetermined face masks.

10. A method according to claim 9, wherein the one of the plurality of predetermined face masks that comprises geometry that most closely corresponds to the estimated face size and/or the determined Gaussian curve is selected from the plurality of face masks.

11. A method according to claim 10, comprising performing regression analysis to determine the one of the plurality of predetermined face masks.

12. A method according to claim 9, further comprising determining whether the estimated face size of the user and the determined Gaussian curve are within a threshold range.

13. A method according to claim 12, wherein the one of the plurality of predetermined face masks is selected if the estimated face size of the user and the determined Gaussian curve fall within the threshold range.

14. A method according to claim 12, wherein if the estimated face size of the user and the determined Gaussian curve fall outside of the threshold range, one of the plurality of predetermined face masks is not selected, and the method further comprises selecting the parameters for the profile geometry of the face mask using geometry defined by the point cloud.

15. A method according to claim 14, wherein selecting the parameters for the profile geometry comprises modifying parameters of one of the plurality of predetermined face masks based on the geometry defined by the point cloud.

16. A method according to claim 9, wherein selecting the one of the plurality of predetermined face masks comprises selecting one of a plurality of predetermined main bodies and/or selecting one of a plurality of predetermined seals.

17. A method according to claim 1, wherein determining the best-fit Gaussian curve comprises determining a width and height of the profile of the nose, based on the facial data, and selecting the parameters of the Gaussian curve based on the determined width and height.

18-22. (canceled)

23. A system for determining a geometry of a face mask comprising a main body and a seal, the seal configured to engage a nasal region, cheeks and a chin of a user, the face mask configured to provide the user with respiratory protection, the system comprising:
a facial data collector configured to collect facial data of the user comprising a point cloud comprising a plurality of points on a skin surface in a nasal region, an eye region and a chin region of the user; and
an apparatus comprising:
a receiver configured to receive the facial data from the facial data collector device;
a face size estimator configured to determine, based on the facial data comprising the plurality of points in at least one of the eye region and the chin region of the user, an estimated face size of the user;
a nasal profile determiner configured to determine, based on the facial data comprising the plurality of points of the nasal region, a best-fit Gaussian curve to a profile of a nose of the user; and
a mask geometry determination unit configured to select parameters for a profile geometry of the face mask using the estimated face size of the user and the determined Gaussian curve.

24. A computer program comprising instructions which, when executed on at least one processor, cause the at least one processor to carry out the method according to claim 1.

25. A face mask comprising a main body and a seal, the seal configured to engage a nasal region, cheeks and a chin of a user, the face mask configured to provide the user with respiratory protection, the face mask obtainable by the method according to claim 1.
